(19) United States
(12) Patent Application Publication
(10) Pub. No.: US 2025/0086819 A1
(43) Pub. Date: Mar. 13, 2025

(54) METHOD AND SYSTEM FOR TRACKING MARKER IN AUGMENTED REALITY

(71) Applicant: Decasight Corporation, Seoul (KR)

(72) Inventors: Byung Joon PARK, Seoul (KR); Soo Ho CHOI, Gyeonggi-do (KR); Tae Hyun KIM, Seoul (KR); Ji Ho PARK, Seoul (KR); Ki Wook LEE, Incheon (KR)

(21) Appl. No.: 18/038,322

(22) PCT Filed: Mar. 28, 2023
(86) PCT No.: PCT/KR2023/004075

§ 371 Date: May 23, 2023

(30) Foreign Application Priority Data
Mar. 24, 2023 (KR) 10-2023-0038708

(51) Int. Cl. G06T 7/70; G06T 19/00

(52) U.S. Cl. CPC G06T 7/70; G06T 19/006

(57) ABSTRACT

Disclosed is a marker tracking method in a marker tracking system for tracking a marker in augmented reality, the method including a process of creating marker information necessary for estimating a pose of the marker, and a process of estimating the pose of the marker on the basis of the marker information. By providing the marker tracking method in augmented reality using dense image alignment, it is possible to guarantee high tracking accuracy even with a single camera and eliminate any restriction in the tracking area in the case of using a plurality of cameras.

### CROSS REFERENCES TO RELATED APPLICATIONS

The present invention contains subject matter related to Korean Patent Application No. 10-2023-0038708, filed in the Korean Patent Office on Mar. 24, 2023, the entire contents of which are incorporated herein by reference.

### BACKGROUND

### Field of the Invention

The present invention relates to a marker tracking technology, and more particularly, to a marker tracking technology in augmented reality frequently used for medical purposes.

### Description of Related Art

Augmented Reality (AR) is known as a technology capable of combining the digital world with the real world to make possible things that were impossible in reality. Recently, AR technology is being used in various types of utilizations such as surgical navigation in the medical field.

In relation to surgery, virtual reality and augmented reality technologies are currently applied to virtual endoscopy, image guided surgery, preoperative planning, and the like. In application of virtual endoscopy, a target part of the patient's body is examined while virtually exploring the internal organs on a volume image received through imaging such as MRI (Magnetic Resonance Imaging) or CT (Computed Tomography) in a virtual space. Endoscopy is most frequently applied to the stomach and large intestine. Virtual endoscopy has an image quality not lower than that of actual endoscopy, and burdens no pain to the patient because it is performed in a virtual space. Moreover, since virtual endoscopy can be applied to blood vessels or cerebral cavities that are difficult to actually explore, it is expected to be widely used in diagnosis in the future. Image-guided surgery is based on augmented reality rather than virtual reality, and it enables accurate surgery by showing the inside of the area around the part to be operated by matching it with the actual part. Pre-surgery planning is a technology that helps to plan in advance which method is most effective for surgery by classifying, visualizing, and manipulating the patient's organs or tissues in a virtual space before surgery.

Currently, an electromagnetic tracking method and a stereoscopic optical tracking method are most commonly used in tracking for medical purposes. However, both methods are not suitable for the AR environments where small devices such as a HMD (Head Mounted Display) are utilized due to their structural characteristics.

A square-shaped fiducial marker tracking method is widely used in AR environments using small devices. However, conversely, it is not suitable for the medical purpose due to low accuracy.

The electromagnetic tracking method is known as a conventional medical marker tracking method. In this method, an electromagnetic field is generated from a field generator (FG), and location recognition is performed on the basis of the electric current induced in a sensor. Since the electromagnetic field can penetrate the human body, tracking is possible even when a sensor attached to a tip of the tool enters the human body. However, when there is any factor that affects the electromagnetic field in the surroundings, the accuracy is degraded. Since the electromagnetic field becomes weaker as it recedes from field generator, the accuracy decreases as the sensor position becomes farther away. In order to utilize electromagnetic tracking in the AR environment, alignment between an AR camera and an EM coordinate system is required. Since a separate optical marker is used in this process, a tracking error in this process is inevitably added to the total error disadvantageously.

In the stereoscopic optical tracking system as a conventional marker tracking method in the medical field, a spherical IR marker tracking system and an infrared camera are utilized, and tracking can be performed by simultaneously shooting the marker with two or more cameras. In this technique, two or more cameras are necessary, and tracking can be made only when at least three IR markers are visible on the shared field of view of the cameras. Therefore, there is a limitation due to the distance between the cameras.

FIG. 1 shows tracking areas of cameras in a conventional stereoscopic optical tracking method.

Referring to FIG. 1, when the distance between cameras is relatively long, the tracking area becomes narrow, and depth estimation accuracy increases. In contrast, when the distance is relatively short, the tracking area becomes wide, and depth estimation accuracy decreases.

FIG. 2 shows a marker displayed on the AR screen in the fiducial marker tracking method. In FIG. 2, a flat fiducial marker tracking method is illustrated, in which the marker is displayed on the AR screen as a result of inaccurate object matching.

As shown in FIG. 2, among conventional general-purpose marker tracking methods, the fiducial marker tracking technique utilizes a system for estimating a pose of the fiducial marker captured by the camera. In this technique, tracking can be performed with only a single camera, and thus, it is easy to use in small devices. However, it is difficult to use for medical purposes due to low tracking accuracy. In addition, shaking may occur in the AR content, which may reduce the user's AR immersion feeling disadvantageously.

### SUMMARY

In view of the aforementioned problems, it is therefore an object of the invention to provide a method and system for tracking markers in augmented reality on the basis of dense image alignment.

The object of the present invention is not limited to the purpose mentioned above, and other objects not mentioned will become apparent to those skilled in the art by reading the following description.

According to an aspect of the present invention, there is provided a marker tracking method in a marker tracking system for tracking a marker in augmented reality, the method comprising: creating marker information necessary for marker pose estimation; and estimating a marker pose on the basis of the marker information.

In the marker tracking method described above, assuming that an aggregate of one or more individual fiducial markers is called a marker set, the process of creating the marker information may include creating a fiducial marker by setting the fiducial marker to be used, creating 3D information of the fiducial marker, and performing surface calibration for the fiducial marker.

In the marker tracking method described above, the process of performing surface calibration for the fiducial marker may include acquiring a pose of a marker set from each image taken by photographing the marker set at various angles from one or more cameras, and optimizing the pose of the marker set and poses of individual fiducial markers for each image on the basis of a nonlinear optimization algorithm.

In the marker tracking method described above, the process of estimating the marker pose may include a marker corner information collecting step for collecting information on a corner of the marker, a marker initial pose estimation step for estimating an initial pose of the marker on the basis of the collected corner information, a marker coarse pose refinement step for adjusting the initial pose of the marker so as to minimize an error (reprojection error) between 2D corner coordinates of the markers obtained through the marker corner information collecting step and 2D coordinates obtained by reprojecting 3D corner coordinates on the world coordinate system, and a marker fine pose refinement step for readjusting a detailed pose of the marker on the basis of a dense image alignment technique so as to minimize an appearance difference between the detected fiducial marker image and a template fiducial marker image prepared in advance.

In the marker tracking method described above, the dense image alignment technique may include the Lucas-Kanade image alignment.

According to another aspect of the present invention, there is provided a marker tracking system for tracking a marker in augmented reality, the system comprising: one or more cameras provided to photograph a marker set as an aggregate of one or more individual fiducial markers; and a computing device configured to obtain an image captured by the camera, create marker information necessary for estimating a marker pose from the obtained image, and estimate the marker pose on the basis of the marker information.

In the marker tracking system described above, the computing device may be configured to, in the process of creating the marker information, create a fiducial marker by setting the fiducial marker to be used, create 3D information of the fiducial marker, and perform surface calibration for the fiducial marker.

In the marker tracking system described above, the computing device may be configured to, in the process of performing surface calibration for the fiducial marker, acquire a pose of the marker set from each image obtained by photographing the marker set at various angles from one or more cameras, and optimize the pose of the marker set and the poses of individual fiducial markers for each image on the basis of a nonlinear optimization algorithm.

In the marker tracking system described above, the computing device may be configured to, in the process of estimating the marker pose, collect information on corners of the marker, estimate an initial pose of the marker on the basis of the collected corner information, adjust the initial pose of the marker so as to minimize an error (reprojection error) between 2D corner coordinates of the markers obtained through the marker corner information collecting process and 2D coordinates obtained by reprojecting 3D corner coordinates on the world coordinate system, and readjust a detailed pose of the marker on the basis of a dense image alignment technique so as to minimize an appearance difference between the detected fiducial marker image and a template fiducial marker image prepared in advance.

In the marker tracking system described above, the dense image alignment technique may include the Lucas-Kanade image alignment technique.

According to the present invention, a marker tracking method in augmented reality using dense image alignment is provided. Therefore, it is possible to guarantee high tracking accuracy with only a single camera, and eliminate restriction in the tracking area in the case of using a plurality of cameras.

In addition, according to the present invention, since there are many fiducial markers that can be obtained within a shared field of view from a plurality of cameras, it is possible to perform more precise pose tracking.

### BRIEF DESCRIPTION OF THE DRAWINGS

The foregoing and additional features and characteristics of this disclosure will become more apparent from the following detailed description considered with reference to the accompanying drawings, wherein:

FIG. 1 illustrates tracking areas of cameras in a conventional stereoscopic optical tracking method;

FIG. 2 illustrates a marker displayed on an AR screen in a fiducial marker tracking method;

FIG. 3 schematically illustrates a configuration of a marker tracking system according to an embodiment of the present invention;

FIG. 4 illustrates tracking areas of cameras in a marker tracking method according to an embodiment of the present invention;

FIG. 5 is a flowchart illustrating a marker tracking method in augmented reality according to an embodiment of the present invention;

FIG. 6 is a flowchart illustrating a detailed process of the marker tracking method in augmented reality according to an embodiment of the present invention;

FIG. 7 illustrates a fiducial marker, a marker set, and indices;

FIG. 8 illustrates marker types;

FIG. 9 illustrates marker set types;

FIG. 10 illustrates states of the marker set around surface calibration;

FIG. 11 illustrates individual fiducial markers and a marker set;

FIG. 12 shows photographs of a marker set taken at various angles;

FIG. 13 is a flowchart illustrating a surface calibration method according to an embodiment of the present invention;

FIG. 14 illustrates a marker corner detection course according to an embodiment of the present invention;

FIGS. 15, 16, 17 and 18 are diagrams for explaining a marker corner tracking course according to an embodiment of the present invention;

FIG. 19 is a flowchart illustrating an initial pose estimation course according to an embodiment of the present invention;

FIGS. 20 and 21 are exemplary diagrams for explaining an initial pose estimation course according to an embodiment of the present invention;

FIG. 22 is a flowchart illustrating a coarse pose refinement course according to an embodiment of the present invention;

FIG. 23 is an exemplary diagram for explaining a fine pose refinement course according to an embodiment of the present invention;

FIG. 24 is a flowchart illustrating a fine pose refinement course according to an embodiment of the present invention;

FIGS. 25 and 26 are diagrams for explaining nonlinear optimization; and

FIG. 27 shows schematics of the Lucas-Kanade image alignment algorithm.

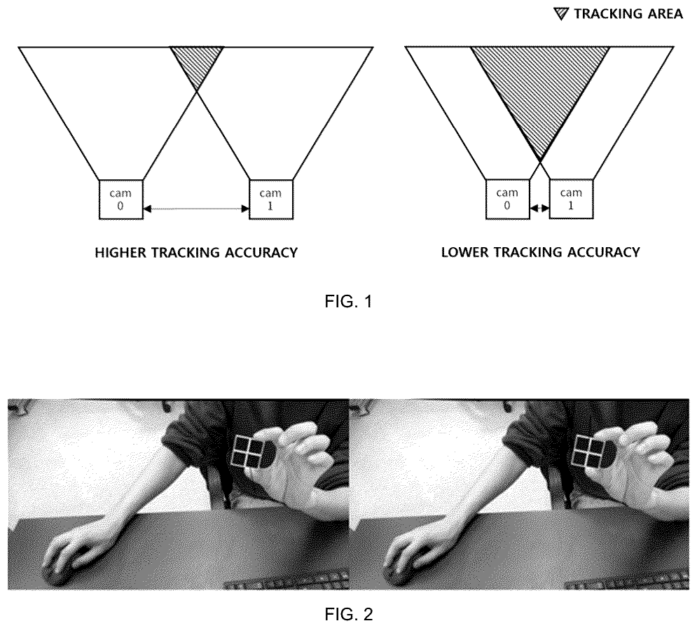

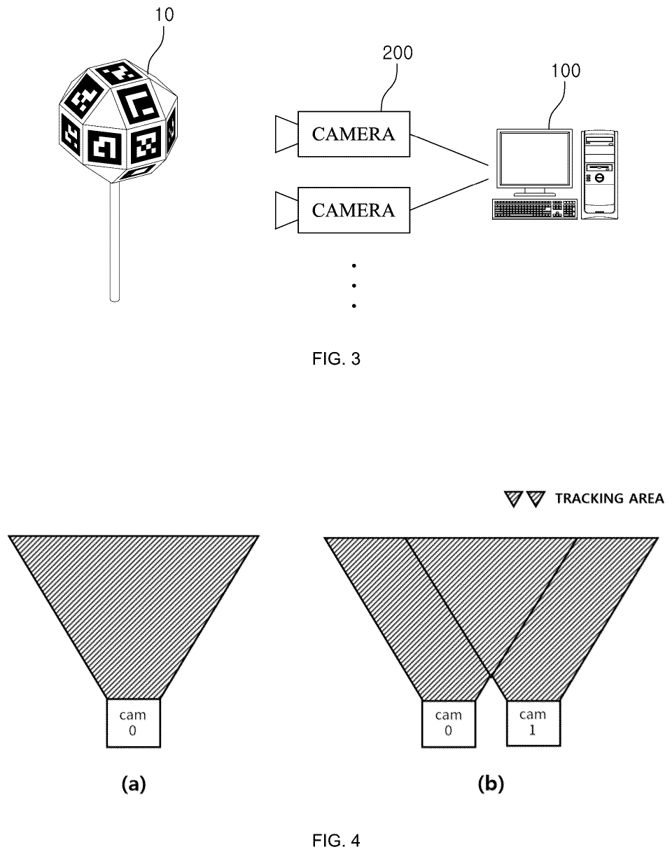

### DETAILED DESCRIPTION

FIG. 3 schematically illustrates a configuration of a marker tracking system according to an embodiment of the present invention.

Referring to FIG. 3, a marker tracking system according to an embodiment of the present invention includes a computing device 100 and a camera 200.

The computing device 100 tracks the marker in the augmented reality image photographed by the camera 200.

The camera 200 photographs a marker set 10, which is an aggregate of one or more individual fiducial markers. According to the present invention, one or more cameras 200 may be provided.

According to the present invention, the computing device 100 may track a fiducial marker on the basis of dense image alignment.

The computing device 100 acquires images photographed by the cameras 200 for every frame in real time.

In addition, the computing device 100 creates marker information necessary for estimating the marker pose as a preliminary task before the marker tracking.

In addition, the computing device 100 may estimate the marker pose on the basis of the marker information. The computing device 100 repeats the marker pose estimation process on the basis of the image captured by the camera 200 and the marker information until a command for terminating execution is received from a user.

In creating the marker information, the computing device 100 may set a fiducial marker to be used, create the fiducial marker and 3D information of the fiducial marker, and perform surface calibration for the fiducial marker.

In performing surface calibration for the fiducial marker, the computing device 100 may acquire the pose of the marker set from each image obtained by photographing the marker set at various angles from one or more cameras, and optimize the pose of the marker set and the poses of individual fiducial markers for each image on the basis of a nonlinear optimization algorithm. That is, according to the present invention, a plurality of poses of the marker set may be obtained by taking a plurality of pictures either from a single camera or from a plurality of cameras.

In estimating the marker pose, the computing device 100 may collect information on corners of the markers, estimate the initial marker pose on the basis of the collected information on the corners, adjust the initial marker pose so as to minimize an error (reprojection error) between the 2-dimensional coordinates of the markers obtained through the process of collecting the information on the corners of the markers described above and the 2-dimensional coordinates obtained by reprojecting the 3-dimensional coordinates of the corners on the world coordinate system, and readjust the detailed marker pose on the basis of dense image alignment so as to minimize an appearance difference between the detected fiducial marker image and a template fiducial marker image prepared in advance.

Specific embodiments described herein are representative of preferable implementations or exemplifications of the present invention, and the scope of the invention is not limited thereby. Those skilled in the art would appreciate that further modifications and applications may be possible without departing from the spirit and scope of the invention as defined in claims and their equivalents.

The terminology used herein is only for the purpose of describing particular embodiments and is not intended to limit the invention. As used herein, the singular forms “a,” “an,” and “the” may be intended to include the plural forms as well, unless the context clearly indicates otherwise. It is further to be noted that, as used herein, the terms “comprises,” “comprising,” “include,” and “including” indicate the presence of stated features, integers, steps, operations, units, and/or components, but do not preclude the presence or addition of one or more other features, integers, steps, operations, units, and/or components, and/or combination thereof.

Unless specified otherwise, all terminologies used herein including technical or scientific terminologies have the same meanings as those generally appreciated by a person ordinarily skill in the art to which the present invention pertains. Terminologies defined in typical dictionaries should be construed to have meanings matching those described in the context of the related art, and should not be construed as being abnormal or excessively formal unless defined apparently herein.

The present invention will now be described with reference to the accompanying drawings, in which like reference numerals denote like elements throughout the entire specification, and they will not be repeatedly described intentionally. In the following description, any specific word or sentence for the related art will not be provided for simplicity purposes if it unnecessarily obscures the subject matter of the invention.

The present invention relates to a marker tracking method in augmented reality.

According to the present invention, a subject performing the marker tracking method in augmented reality may be a marker tracking system or a general computing device that performs the marker tracking method in augmented reality, or may be a controller or processor that controls a system or device(s) that performs the marker tracking method in augmented reality. That is, the marker tracking method in augmented reality according to the present invention may be implemented as an algorithm, which is a kind of software, and the software may be executed in a controller or processor of the marker tracking system or a general computing device that executes the marker tracking method in augmented reality.

The present invention relates to a marker tracking method in a marker tracking system for tracking a marker in augmented reality, and the method includes a process of creating marker information necessary for estimating the marker pose, and a process of estimating a marker pose on the basis of the marker information.

Assuming that an aggregate of one or more individual fiducial markers is called a “marker set”, the process of creating the marker information may include setting a fiducial marker to be used, creating the fiducial marker, creating 3D information of the fiducial marker, and performing surface calibration for the fiducial marker.

The surface calibration for the fiducial marker includes acquiring a pose of the marker set from each image of the marker set taken at various angles from one or more cameras, and optimizing the pose of the marker set and the poses of individual fiducial markers for each image on the basis of a nonlinear optimization algorithm.

The process of estimating the marker pose may include a marker corner information collecting step for collecting information on marker corners, an initial marker pose estimation step for estimating the initial marker pose on the basis of the collected marker corner information, a coarse marker pose refinement step for adjusting the initial marker pose so as to minimize an error (reprojection error) between the 2D corner coordinates of the markers obtained through the marker corner information collecting step and the 2D coordinates obtained by reprojecting the 3D corner coordinates on the world coordinate system, and a fine marker pose refinement step for readjusting the detailed marker pose on the basis of a dense image alignment technique so as to minimize an appearance difference between the detected fiducial marker image and a template fiducial marker image prepared in advance.

The dense image alignment technique may include a Lucas-Kanade image alignment technique.

FIG. 3 schematically illustrates a configuration of a marker tracking system according to an embodiment of the present invention.

Referring to FIG. 3, the marker tracking system according to an embodiment of the present invention includes a computing device 100 and a camera 200.

The computing device 100 tracks the markers in the augmented reality image taken by the camera 200.

The camera 200 photographs a marker set 10 which is an aggregate of one or more individual fiducial markers. According to the present invention, one or more cameras 200 may be provided.

According to the present invention, the computing device 100 may track the fiducial marker on the basis of dense image alignment.

The computing device 100 acquires images captured by the camera 200 for every frame in real time.

In addition, the computing device 100 creates marker information necessary for estimating the marker pose as a preliminary task before the marker tracking.

In addition, the computing device 100 may estimate the marker pose on the basis of the marker information. The computing device 100 repeats the process of estimating the marker pose from the image captured by the camera 200 on the basis of the marker information until a user's end request.

In the process of creating marker information, the computing device 100 may set a fiducial marker to be used, create the fiducial marker, create 3D information of the fiducial marker, and perform surface calibration for the fiducial marker.

In the surface calibration for the fiducial marker, the computing device 100 may acquire the pose of the marker set from each image obtained by photographing the marker set at various angles from one or more cameras, and optimize the pose of the marker set and the poses of individual fiducial markers for each image on the basis of a nonlinear optimization algorithm. That is, according to the present invention, a plurality of marker set poses may be obtained by taking a plurality of pictures either from a single camera or from a plurality of cameras.

In the process of estimating the marker pose, the computing device 100 may collect information on the marker corners, estimate the initial marker pose on the basis of the collected corner information, adjust the initial marker pose so as to minimize an error (reprojection error) between the 2D corner coordinates of the markers obtained through the marker corner information collecting step and the 2D coordinates obtained by reprojecting the 3D corner coordinates on the world coordinate system, and readjust the detailed marker pose on the basis of a dense image alignment technique so as to minimize an appearance difference between the detected fiducial marker image and a template fiducial marker image prepared in advance.

FIG. 4 illustrates a tracking area of a camera in a marker tracking method according to an embodiment of the present invention. FIG. 4(a) shows a tracking area from a single camera, and FIG. 4(b) shows tracking areas from a pair of cameras.

As shown in FIG. 4, according to the present invention, even a single camera can guarantee high tracking accuracy. Even when a plurality of cameras are used, there is no restriction on the tracking area. In addition, since a plurality of fiducial marker corner coordinates can be obtained within the shared field of view, it is possible to perform more precise pose tracking.

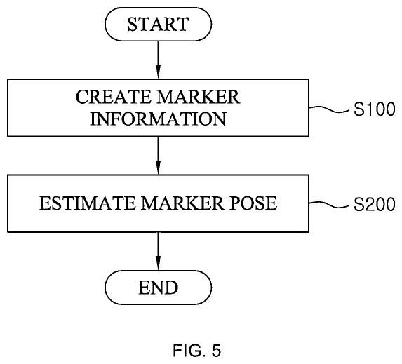

FIG. 5 is a flowchart illustrating a marker tracking method in augmented reality according to an embodiment of the present invention.

Referring to FIG. 5, the marker tracking method in augmented reality according to an embodiment of the present invention includes a process of creating marker information (S100) necessary for estimating a marker pose and a process of estimating the marker pose (S200) on the basis of the marker information.

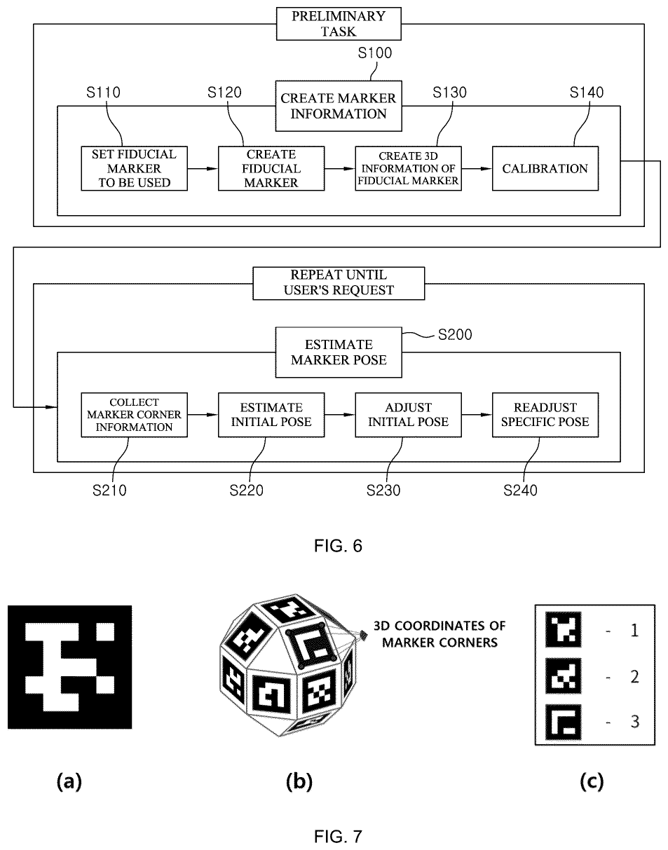

FIG. 6 is a flowchart illustrating details of the marker tracking method in augmented reality according to an embodiment of the present invention.

Referring to FIG. 6, the process of creating the marker information (S100) includes setting a fiducial marker to be used (S110), creating the fiducial marker (S120), creating 3D information of the fiducial marker (S130), and performing surface calibration for the fiducial marker (S140).

The process of estimating the marker pose (S200) includes a marker corner information collecting step S210, a marker initial pose estimation (IPE) step S220, a marker coarse pose refinement (CPR) step S230, and a marker fine pose refinement (FPR) step S240.

In the process of creating the marker information (S100), as a preliminary task before marker tracking, the computing device 100 creates marker information necessary for estimating the marker pose.

In the process of estimating the marker pose (S200), the computing device 100 may estimate the marker pose on the basis of the marker information. In this case, the computing device 100 repeats the process of estimating the marker pose from the image captured by the camera 200 on the basis of the marker information until a user's end request.

In the process of creating the marker information (S100) according to the present invention, various types of preliminary information necessary for estimating the marker pose are created. The types of the information created in this course may include a type of fiducial marker to be used, a type of the marker set formed by the fiducial marker, an index representing marker pattern matching information, and 3D coordinates of marker corners expressed in the unit of the real world.

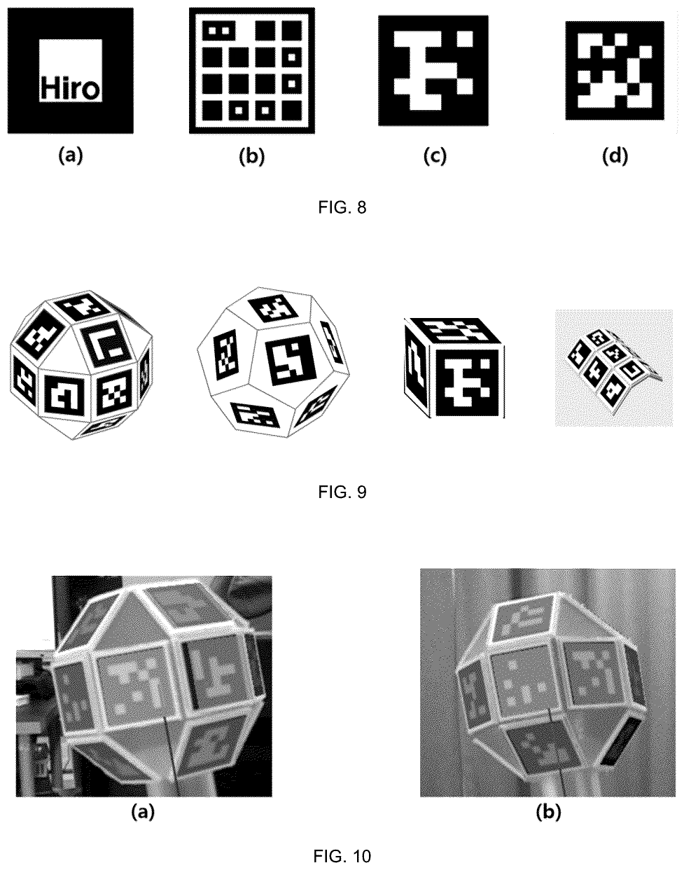

FIG. 7 illustrates examples of the fiducial marker, the marker set, and the indexes.

Specifically, FIG. 7(a) illustrates a fiducial marker, FIG. 7(b) illustrates a marker set as an aggregate of the fiducial markers, and FIG. 7(c) illustrates indexes for representing marker pattern matching information.

According to the present invention, any type of pattern can be employed as long as it is a square-shaped fiducial marker. More precise pose estimation can be made for the fiducial markers having more complex patterns.

FIG. 8 illustrates marker types.

In FIG. 8, types of the fiducial markers usable according to the present invention are illustrated, in which FIG. 8(a) shows an ARToolKit marker, FIG. 8(b) shows a Topo Tag marker, FIG. 8(c) shows an ArUco marker, and FIG. 8(d) shows an AprilTag marker.

As a marker set type usable according to the present invention, any type of markers or any type of their combinations are possible as long as all of the individual fiducial markers maintain a plane. In addition, as long as only two or more fiducial markers are tracked out of the marker set, performance is not significantly affected even when a part of the marker set is covered. In this manner, various types of marker sets can be employed according to the present invention, and can be utilized in various fields as long as the number and size of attachment surfaces are well considered.

FIG. 9 illustrates various types of marker sets usable according to the present invention.

When the markers and the marker set are produced in reality, various errors occur, such as a printing error or an error caused by attaching the markers to the marker set, unlike digitally created models.

According to the present invention, the surface calibration is a process of applying such a real manufacturing error to the model created in the process of creating the marker information (S100). In the surface calibration, each individual fiducial marker poses of the marker set are adjusted by applying a bundle adjustment technique for non-linear optimization.

FIG. 10 illustrates appearance of the marker set before and after the surface calibration, in which FIG. 10(a) shows a marker set before performing the surface calibration process, and FIG. 10(b) shows a marker set after performing the surface calibration process.

As shown in FIG. 10, any error existing in the individual fiducial marker can be corrected by performing the surface calibration.

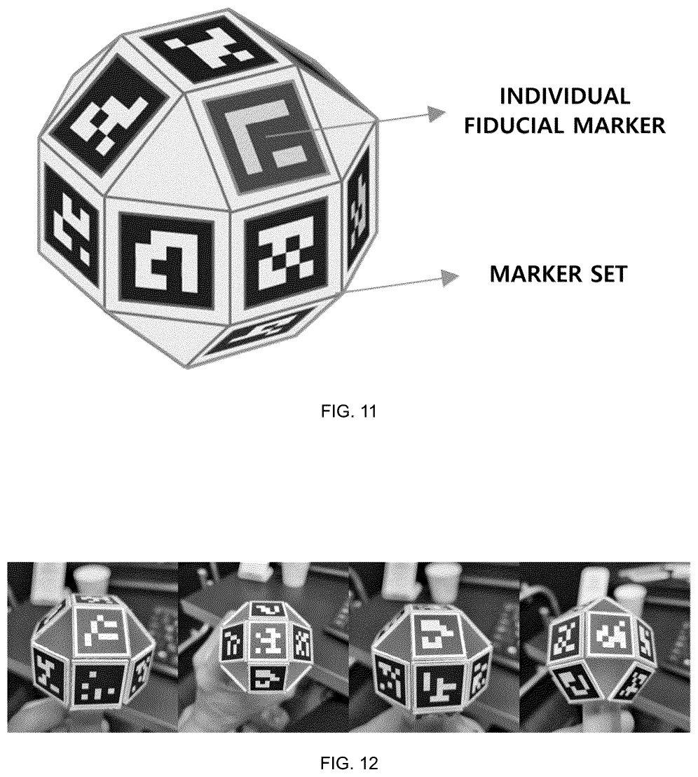

FIG. 11 illustrates individual fiducial markers and a marker set, and FIG. 12 shows photographs of the marker set taken at various angles.

As shown in FIGS. 11 and 12, the “marker set” is a concept including a set of multiple individual fiducial markers.

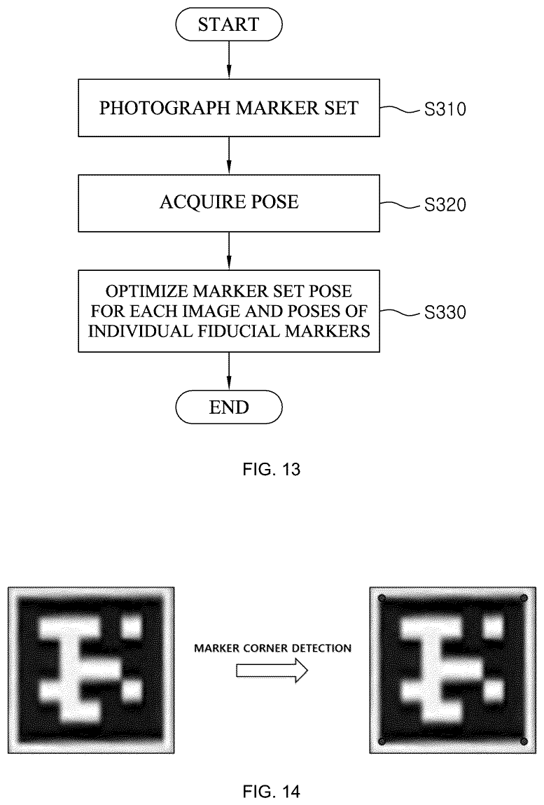

FIG. 13 is a flowchart illustrating a surface calibration method according to an embodiment of the present invention.

Referring to FIG. 13, in the surface calibration method according to an embodiment of the present invention, a marker set is photographed at various angles from one or more cameras (S310), and a pose of the marker set is obtained from each photographed image (S320). FIG. 12 shows examples of the marker set images taken at various angles.

Next, the pose of the marker set and the poses of individual fiducial markers for each image are optimized on the basis of a nonlinear optimization algorithm (S330). In step S330, the pose of the marker set and the poses of individual fiducial markers for each image can be simultaneously optimized on the basis of a nonlinear optimization algorithm such as the Gauss-Newton or Levenberg-Marquardt algorithm.

In this manner, it is possible to more precisely track the marker set by performing the surface calibration according to the present invention.

According to the present invention, surface calibration can be performed so as to minimize an appearance error on the basis of dense image alignment used for the pose estimation.

Here, the object function “E (pⱼ; pₖ)” to be minimized can be expressed as:

$$E ( \{ {p}_{j} ; {p}_{k} \} ) = {\sum }_{i}^{n} {\sum }_{j}^{m} {\sum }_{k}^{I} {( I ( {O}_{proj}^{i} ( {p}_{j} ; {p}_{k} ) ) - T ( {x}_{i} ) )}^{2} ,$$

where “n” denotes the number of individual fiducial markers detected from all of the images;
“m” denotes the number of individual fiducial markers;
“l” denotes the number of photographed images;
“pⱼ” denotes a pose vector of the individual fiducial marker corresponding to the jₜₕ index (6 parameters);
“xᵢ” denotes an index of the iₜₕ marker out of the detected individual fiducial markers;
“Oⁱₚᵣₒⱼ(pⱼ; pₖ)” denotes 2D corner coordinates obtained by reprojecting the 3D corner coordinates of the marker bound with “x” to the pose (pᵢ; pₖ);
“I(Oⁱₚᵣₒⱼ(pⱼ; pₖ))” denotes a brightness vector of all pixels in a square pixel warped from “Oⁱₚᵣₒⱼ(pⱼ; pₖ)”; and
“T(xᵢ)” denotes a brightness vector of all pixels of a template image bound with “xᵢ”.

The actual implementation was performed on the basis of the Gauss-Newton algorithm and the Backtracking Line Search by limiting a threshold value relating to the amount of the appearance error change to 0.01 and limiting the maximum number of iterations to 100 times.

The optimization problem is a problem of adjusting parameters to allow the object function to have an optimal value. In the case of the object function having the first order or lower polynomials, it is a linear optimization problem. In addition, in the case of the second order or higher polynomials, it is a nonlinear optimization problem.

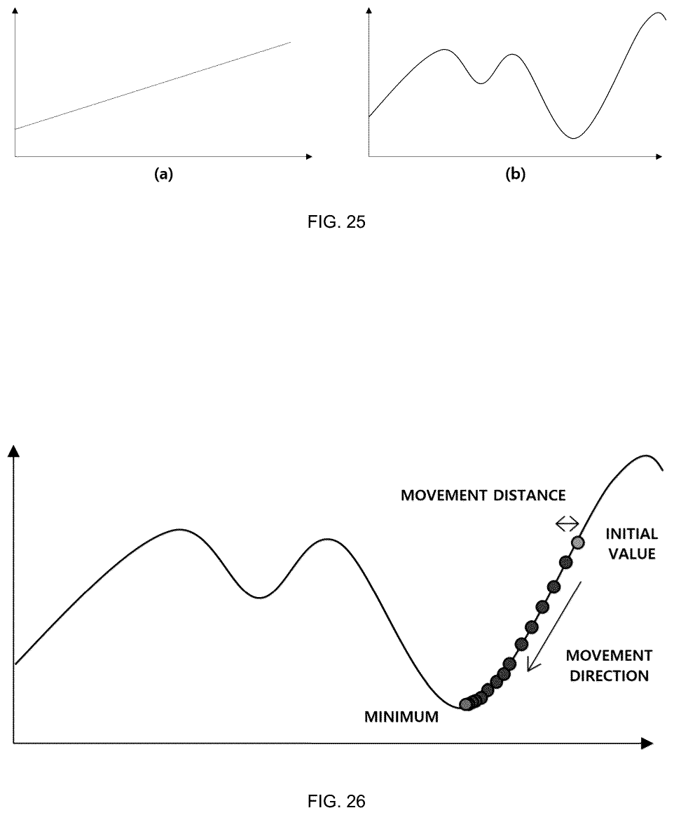

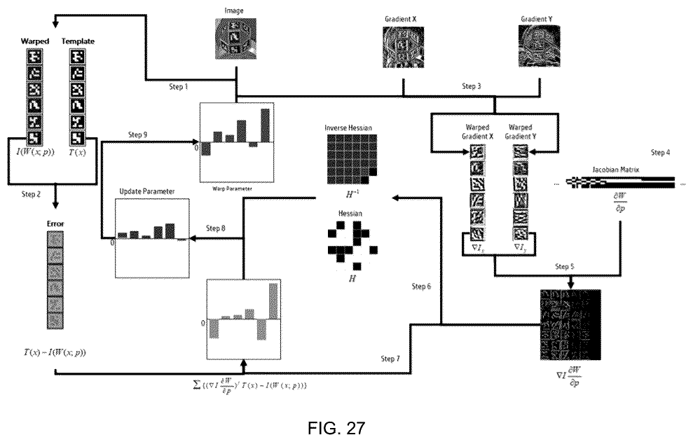

FIGS. 25 and 26 are diagrams for explaining the non-linear optimization.

As shown in FIG. 25, in the linear optimization problem, the shape of the object function is simple. Therefore, it is easy to change the parameters for optimizing the object function. However, in the nonlinear optimization problem, the shape of the object function is complicated. Therefore, it is not easy to change the parameters for optimizing the object function.

The most basic principle for solving the optimization problems is to repeatedly change parameters so as to decrease or increase the function value from the current position. In this case, the most important thing is to set the initial value and determine a movement direction and a movement distance for increasing or decreasing the value. The movement direction is determined on the basis of the first derivative value, and the movement distance is determined on the basis of the second derivative value. FIG. 27 is a graph illustrating a nonlinear optimization process.

There are known various optimization techniques such as Gauss-Newton and Levenberg-Marquardt. However, since most of them are based on the Newton optimization technique, they have the same concept except for the constraints on determining the movement direction and movement distance and their specific calculation methods.

In the actual implementation of the present invention, the Gauss-Newton method was employed.

The Newton optimization method uses the Hessian matrix, which is a second-order derivative matrix of a multivariate vector, to determine whether the current function has reached its maximum or minimum.

The Gauss-Newton method is a method of optimizing an object function using only the Jacobian matrix, which is a first-order derivative matrix of a multivariate vector, instead of the Hessian matrix, in which a pseudo Hessian matrix is created by multiplying the Jacobian matrix and the transposed Jacobian function. With the pseudo Hessian matrix, it is possible to roughly determine whether a local minimum or maximum is present. In the Gauss-Newton method, this process can be repeatedly performed to optimize the value of the object function to be the minimum or maximum.

According to the present invention, the marker corner information collecting step (S210) is a course of collecting 2D corner information from matching pairs of 2D-3D corner information necessary for estimating the pose of the fiducial marker.

The marker corner information collecting step (S210) includes a marker corner detection course for detecting corners of the marker and a marker corner tracking course for tracking a marker corner in the case of failure of the marker corner detection.

FIG. 14 illustrates a marker corner detection course according to an embodiment of the present invention.

As shown in FIG. 14, it is recognized that corners of the marker are detected through the marker corner detection course according to the present invention.

Through the marker corner information collecting step (including thresholding, polygonal approximation, contour detection, corner refinement, and like for the images taken by the camera 200 in step S210), the 2D corner coordinates of the fiducial marker are detected. In this case, any of the marker algorithms known in the art, such as ARToolkit, TopoTag, ArUco, and AprilTag may be employed. In the actual implementation of the present invention, the ArUco detection algorithm was employed, in which an error level was determined on the basis of the image quality.

The marker corner tracking course is a course of compensating for a failure of the marker corner detection, and can be used only when the marker corner detection course for the previous frame succeeds.

In the marker corner tracking course according to the present invention, a position change of the marker corner can be tracked by applying an optical flow to an image of the current frame on the basis of the detection information for the previous frame.

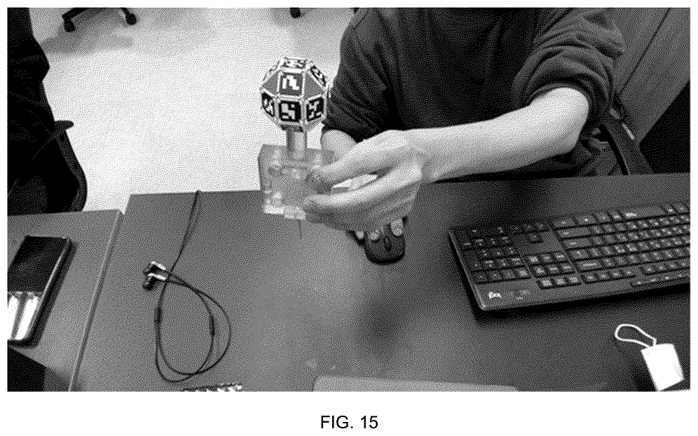

FIGS. 15 to 18 are diagrams for explaining the marker corner tracking course according to an embodiment of the present invention.

Referring to FIGS. 15 to 18, the marker corner tracking according to the present invention is a method of estimating a motion vector between the previous frame and the current frame for the marker corner, in which the marker corner is estimated through three courses.

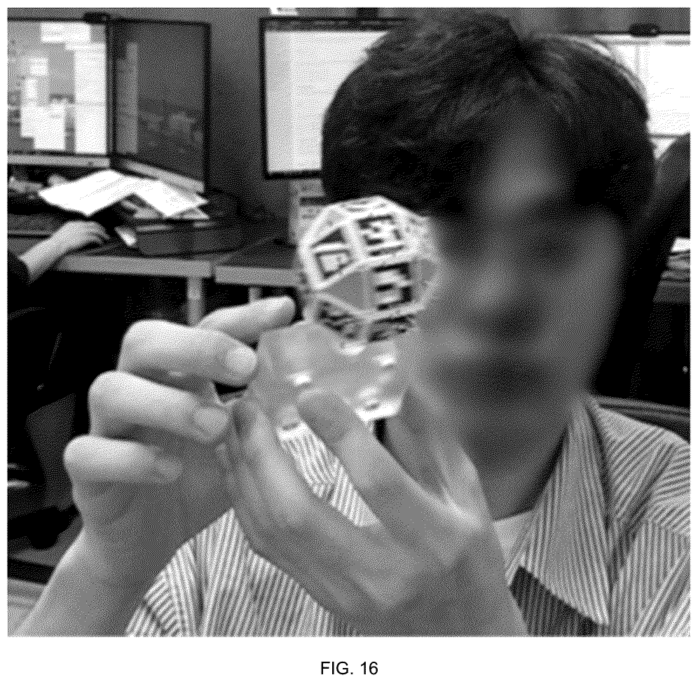

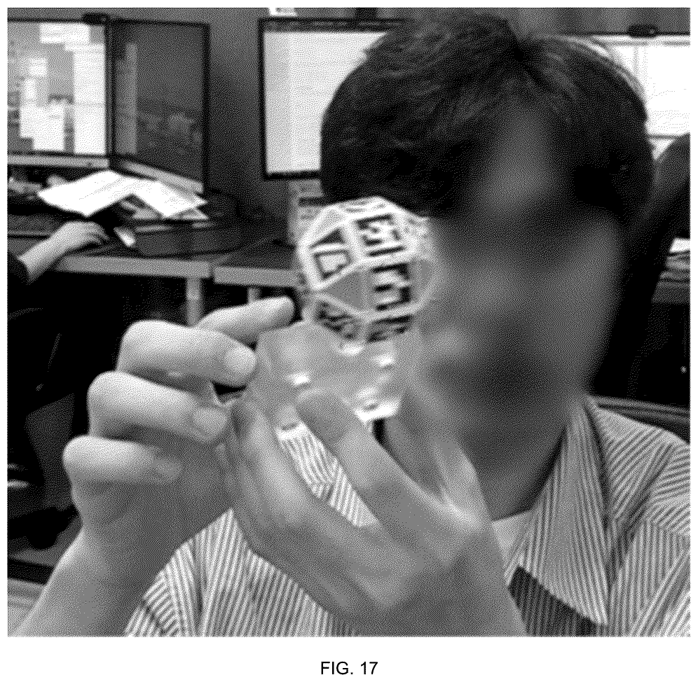

First, direction vectors are created for all marker corners detected from both the previous frame and the current frame. FIG. 16 illustrates an image for creating the direction vector.

Then, the average velocity (a) is obtained from the created direction vectors.

In addition, the velocities of each direction vector are compared with the average velocity (a). When a velocity of the direction vector exceeds 3 times the average (a), the corresponding direction vector is eliminated. FIG. 17 shows an image subjected to primary filtering for the direction vectors.

Subsequently, the average velocity (b) is obtained again from the remaining direction vectors.

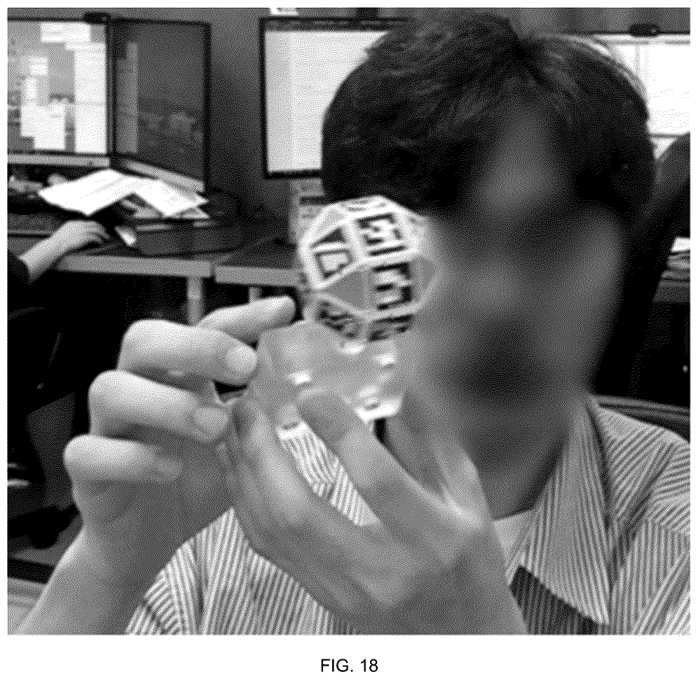

In addition, the velocities of each direction vector are compared with the average velocity (b), and any direction vector whose velocity exceeds 3 times the average (b) is eliminated. As a result, the marker corners can be stably obtained. FIG. 18 shows an image subjected to the secondary filtering for the direction vectors.

In the case of a single camera, the initial marker pose can be obtained by applying the “Perspective N Point” solution to the pairs of 2D coordinates and 3D coordinates obtained through the process of creating the marker information (S100) and the marker corner information collecting step (S210). However, in the case of multiple cameras, it is difficult to apply the same method.

The present invention proposes a method of extending the single-camera-based perspective N point solution to the multiple cameras. Using the method proposed in the present invention, it is possible to estimate the initial pose more precisely from the matching pairs of the 2D-3D coordinates obtained from multiple cameras.

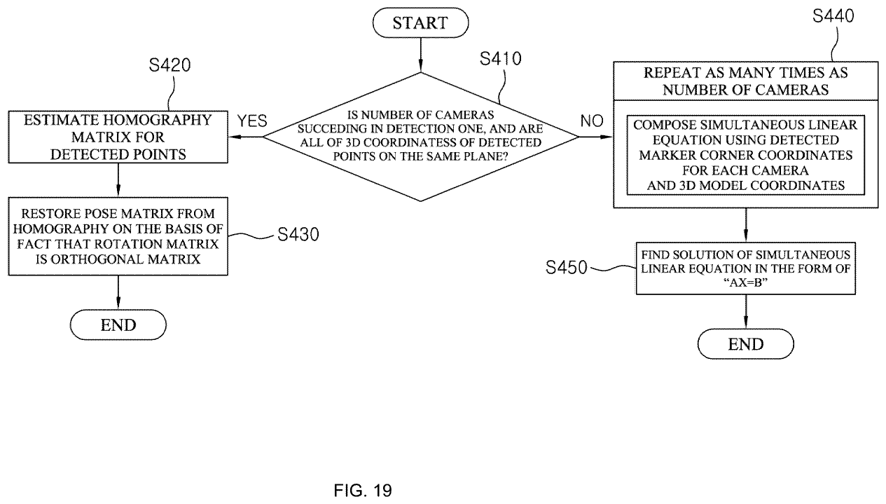

FIG. 19 is a flowchart illustrating an initial pose estimation course according to an embodiment of the present invention.

Referring to FIG. 19, in the initial pose estimation step (S220) according to the present invention, if the number of cameras that have successfully detected the marker corner information is one, and the 3D coordinates of the detected corners are all on the same plane (S410), a homography matrix is estimated for the detected corners (S420). In addition, the pose matrix is restored from the homography on the basis of a fact that the rotation matrix is an orthogonal matrix (S430).

If the number of cameras that have successfully detected the marker corner information is two or more, or if the 3D coordinates of the detected points are not on the same plane (S410), a simultaneous linear equation is composed on the basis of the marker corner coordinates detected from each camera and the 3D model coordinates (S440). In this case, step S440 is repeatedly performed as many times as the number of cameras.

Then, the simultaneous linear equation in the form of “AX=B” is solved (S450).

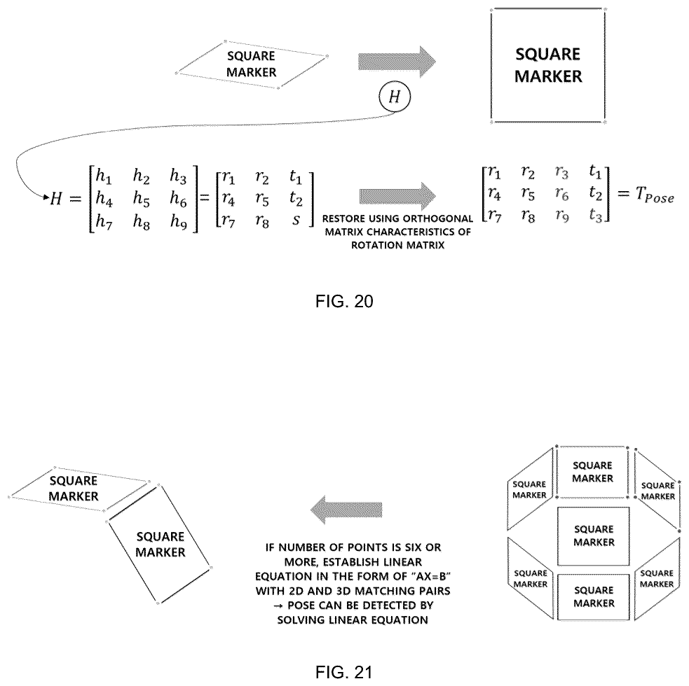

FIGS. 20 and 21 are exemplary diagrams for explaining the initial pose estimation course according to an embodiment of the present invention.

FIG. 20 illustrates a case where the number of cameras that have successfully detected the marker corner information is one, and the 3D coordinates of the detected corners are all on the same plane.

Referring to FIG. 20, a homography matrix H is estimated for the detected corners, and a pose matrix is restored from the homography on the basis of a fact that the rotation matrix is an orthogonal matrix.

The homography matrix (H) can be expressed as follows.

$$H = [ \begin{matrix}{h}_{1} & {h}_{2} & {h}_{3} \\ {h}_{4} & {h}_{5} & {h}_{6} \\ {h}_{7} & {h}_{8} & {h}_{9}\end{matrix} ] = [ \begin{matrix}{r}_{1} & {r}_{2} & {t}_{1} \\ {r}_{4} & {r}_{5} & {t}_{2} \\ {r}_{7} & {r}_{8} & s\end{matrix} ]$$

In addition, the pose matrix Tₚₒₛₑ is restored from the homography on the basis of the orthogonal matrix characteristics of the rotation matrix.

$$[ \begin{matrix}{r}_{1} & {r}_{2} & {r}_{3} & {t}_{1} \\ {r}_{4} & {r}_{5} & {r}_{6} & {t}_{2} \\ {r}_{7} & {r}_{8} & {r}_{9} & {t}_{3}\end{matrix} ] = {T}_{Pose}$$

FIG. 21 illustrates a case where the number of cameras that have successfully detected the marker corner information is two or more, or the 3D coordinates of detected points are not on the same plane.

Referring to FIG. 21, a simultaneous linear equation is composed using the marker corner coordinates detected for each camera and 3D model coordinates, and the simultaneous linear equation in the form of “AX=B” is solved. If there are more than six points, a linear equation in the form “AX=B” is established with the 2D-3D matching pairs, and the initial marker pose can be estimated by solving this linear equation.

According to the present invention, if the number of cameras that have successfully detected the marker corner information is two or more, or the 3D coordinates of the detected points are not on the same plane, the initial pose estimation course can be expressed as the following formulas.

The model-world coordinate system transformation matrix T_{world} is obtained by solving the simultaneous linear equation by expanding the direct linear transform technique.

$${U}_{img}^{ij} = [ \begin{matrix}{u}^{ij} \\ {v}^{ij} \\ 1\end{matrix} ]$$

$${C}_{int}^{i} = [ \begin{matrix}{f}_{x}^{i} & 0 & {c}_{x}^{i} \\ 0 & {f}_{y}^{i} & {c}_{y}^{i} \\ 0 & 0 & 1\end{matrix} ]$$

$${C}_{ext}^{i} = [ \begin{matrix}{r}_{1}^{i} & {r}_{2}^{i} & {r}_{3}^{i} & {t}_{1}^{i} \\ {r}_{4}^{i} & {r}_{5}^{i} & {r}_{6}^{i} & {t}_{2}^{i} \\ {r}_{7}^{i} & {r}_{8}^{i} & {r}_{9}^{i} & {t}_{3}^{i}\end{matrix} ]$$

$${C}_{ext}^{j} = [ \begin{matrix}{r}_{1}^{i} & {r}_{2}^{i} & {r}_{3}^{i} & {t}_{1}^{i} \\ {r}_{4}^{i} & {r}_{5}^{i} & {r}_{6}^{i} & {t}_{2}^{i} \\ {r}_{7}^{i} & {r}_{8}^{i} & {r}_{9}^{i} & {t}_{3}^{i}\end{matrix} ]$$

$${P}_{model}^{ij} = [ \begin{matrix}{x}^{ij} \\ {y}^{ij} \\ {z}^{ij} \\ 1\end{matrix} ]$$

where “U^{ij}_{img}” denotes jₜₕ 2D corner coordinates detected from the in camera;
“Cⁱᵢₙₜ” denotes an intrinsic parameter of the in camera;
“Cⁱₑₓₜ” denotes an extrinsic parameter of the in camera;
“T_{world}” denotes a model-world coordinate system transformation matrix;
“P^{ij}_{model}” denotes 3D corner coordinates corresponding to U^{ij}_{img};
“u^{ij}, v^{ij}” denotes 2D (u, v) axis (abssisa, ordinate) coordinates of U^{ij}_{img};
“fⁱₓ, fⁱ_{y}” denotes a focal distance of the iₜₕ camera (on the basis of the pixel size in the x-axis and y-axis directions);
“Cⁱₓ, cⁱ_{y}” denotes a center point in the x-axis and y-axis directions of the iₜₕ camera;
“rⁱ_{c}” denotes a cₜₕ factor of the rotation matrix of the iₜₕ camera;
“tⁱ_{c}” denotes a cₜₕ factor of the motion matrix of the iₜₕ camera;
“W_{c}” denotes a cₜₕ factor of T_{world}, and
“x^{ij}, y^{ij}, z^{ij}” denotes 3D (x, y, z) coordinates of P^{ij}_{model}.

$${U}_{norm}^{ij} = {( {C}_{int}^{i} )}^{- 1} {U}_{img}^{ig} = {s [ \begin{matrix}{f}_{x}^{i} & 0 & {c}_{x}^{i} \\ 0 & {f}_{y}^{i} & {c}_{y}^{i} \\ 0 & 0 & 1\end{matrix} ]}^{- 1} [ \begin{matrix}{u}^{ij} \\ {v}^{ij} \\ 1\end{matrix} ] = [ \begin{matrix}{\overline{u}}^{ij} \\ {\overline{v}}^{ij} \\ 1\end{matrix} ]$$

where “Cⁱᵢₙₜ” denotes an intrinsic parameter of the iₜₕ camera;
“U^{ij}_{img}” denotes jₜₕ 2D corner coordinates detected from the iₜₕ camera;
“U^{ij}ₙₒᵣₘ” denotes normalized coordinates of U^{ij}_{img};
“s” denotes a scale constant;
“u^{ij}, v^{ij}” denotes 2D (u, v) axis (abssisa, ordinate) coordinates of U^{ij}_{img};
“fⁱₓ, fⁱ_{y}” denotes a focal distance of the in camera (on the basis of the pixel size in the x-axis and y-axis directions);
“Cⁱₓ, Cⁱ_{y}” denotes a center point in the x-axis and y-axis directions of the iₜₕ camera; and “ūᵢⱼ, v̅ᵢⱼ” denotes 2D (u, v) axis (abssisa, ordinate) coordinates of U^{ij}ₙₒᵣₘ.

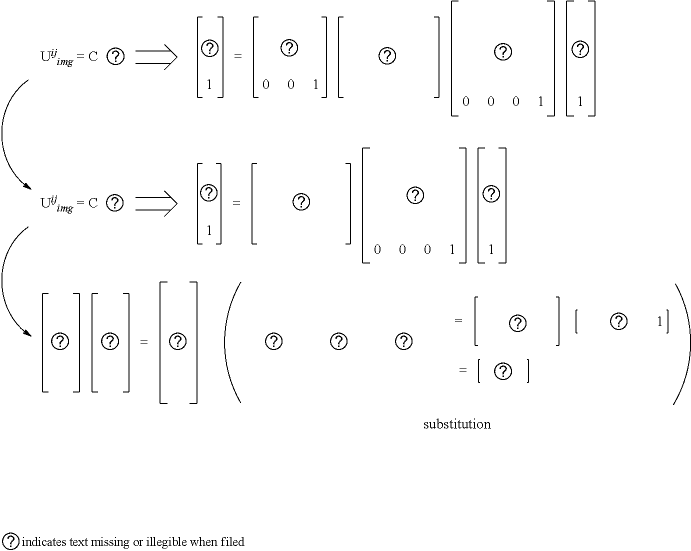

where “U^{ij}_{img}” denotes jₜₕ 2D corner coordinates detected from the iₜₕ camera;
“Cⁱᵢₙₜ” denotes an intrinsic parameter of the iₜₕ camera;
“Cⁱₑₓₜ” denotes an extrinsic parameter of the iₜₕ camera;
“T_{world}” denotes a model-world coordinate system transformation matrix; and
“P^{ij}_{mode}” denotes 3D corner coordinates corresponding to U^{ij}_{img}.

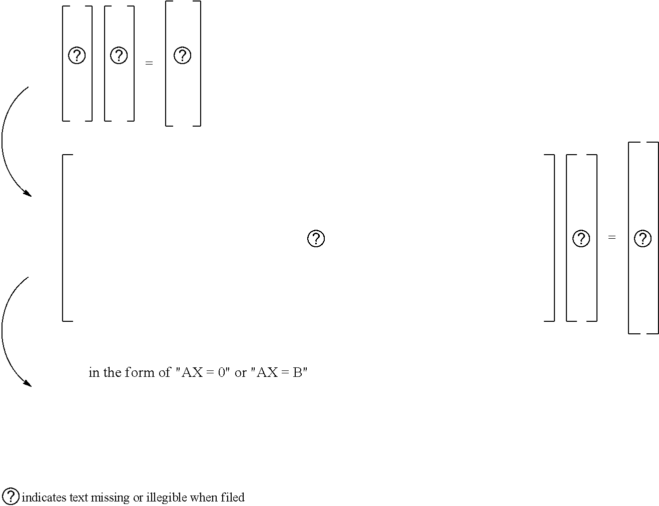

A solution can be derived by various methods such as SVD or Pseudo Inverse for such simultaneous linear equations.

In the coarse pose refinement step S230 according to the present invention, the initial pose is adjusted so as to minimize an error (reprojection error) between the 2D corner coordinates of the markers obtained through the marker corner information collecting step S210 and the 2D coordinates obtained by reprojecting the 3D corner coordinates on the world coordinate system. In step S230 of the present invention, a nonlinear optimization technique such as Gauss-Newton and Levenberg-Marquardt may be employed. In this case, when there is a lot of noise in the marker corner information, the result may deteriorate.

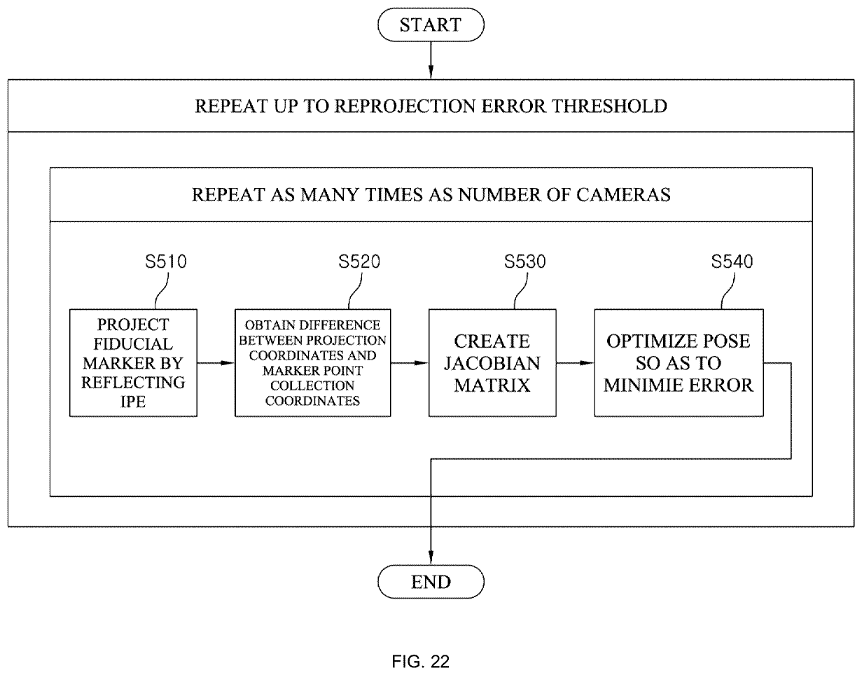

FIG. 22 is a flowchart illustrating a coarse pose refinement course according to an embodiment of the present invention.

Referring to FIG. 22, in the coarse pose refinement step S230 according to an embodiment of the present invention, a fiducial marker is projected by reflecting the result of the initial pose estimation step S220 (S510).

In addition, a difference between the projection coordinates and the marker corner coordinates is obtained (S520), and a Jacobian matrix is created on the basis of this difference (S530).

In addition, the marker pose is optimized by using the Jacobian matrix so as to minimize the error (S540).

In the coarse pose refinement step (S230) according to the present invention, steps S510 to S540 are repeated up to the reprojection error threshold and as many times as the number of cameras.

In the coarse pose refinement step (S230) according to the present invention, nonlinear optimization is performed so as to minimize the reprojection error.

The object function E (p) to be minimized can be expressed as:

$$E ( p ) = \frac{1}{nm} {\sum }_{i = 0}^{n} {\sum }_{j = 0}^{m} {( {O}_{img}^{ij} - {O}_{proj}^{ij} ( p ) )}^{2} ,$$

where “n” denotes the number of cameras;
“m” denotes the number of the detected markers;
“p” denotes a pose vector of the marker set (six parameters);
“O^{ij}_{img}” denotes 2D corner coordinates of the jₜₕ marker detected from the iₜₕ camera; and
“O^{ij}ₚᵣₒⱼ(p)” denotes 2D corner coordinates obtained by reprojecting the 3D corner coordinates of the marker corresponding to “O^{ij}_{img}” to the pose p.

According to the present invention, the actual implementation was performed on the basis of the Levenberg-Marquardt algorithm. The threshold value relating to the reprojection error was the machine error (Machine Epsilon, 1.192092896e-07F), and the maximum number of iterations was limited to 20 times.

According to the present invention, the fine pose refinement step S240 is a course of additionally adjusting the marker pose result value obtained in the coarse pose refinement step S230.

The fiducial marker pose is readjusted on the basis of the dense image alignment technique so as to minimize an appearance difference between the fiducial marker image detected in the fine pose refinement step S240 and a template fiducial marker image prepared in advance.

According to an embodiment of the present invention, the Lucas-Kanade image alignment technique may be employed as the dense image alignment technique.

In the fine pose refinement step S240, the pose adjustment target is not the information obtained in the marker corner information collecting step S210, but the template fiducial marker image. Therefore, even when there is noise in the information obtained in the marker corner information collecting step (S210), its influence is negligible.

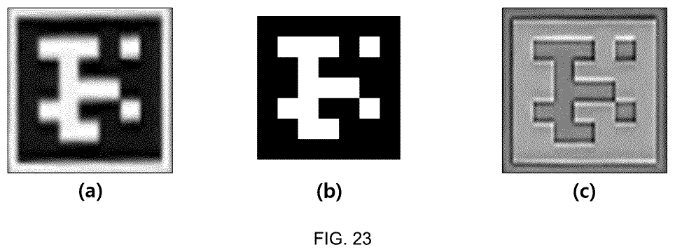

FIG. 23 is an exemplary diagram for explaining the fine pose refinement course according to an embodiment of the present invention.

In FIG. 23, FIG. 23(a) shows a detected fiducial marker image, FIG. 23(b) shows a template fiducial marker image, and FIG. 23(c) shows an appearance difference between the fiducial marker images of FIGS. 23(a) and 23(b).

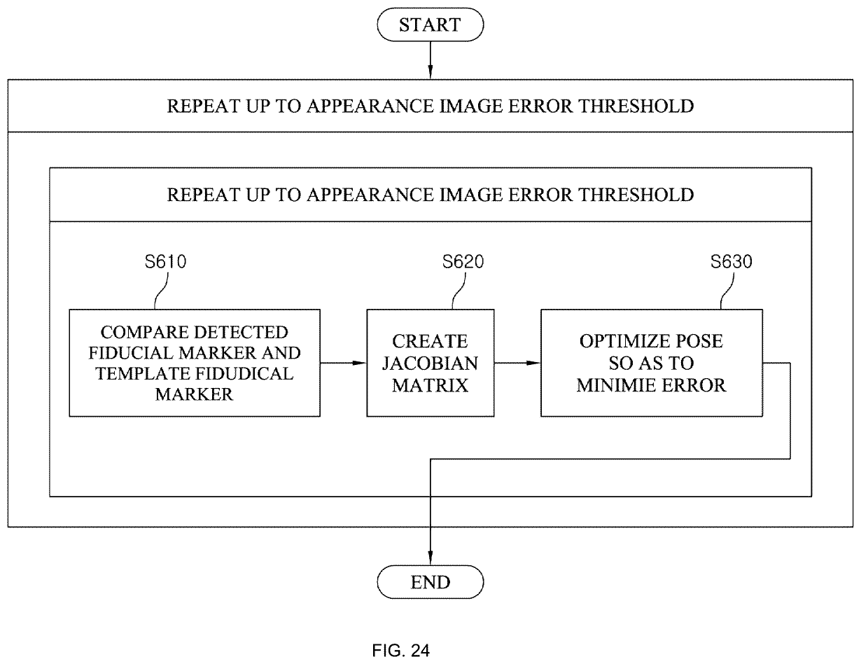

FIG. 24 is a flowchart illustrating a fine pose refinement course according to an embodiment of the present invention.

Referring to FIG. 24, in the fine pose refinement step S240 according to an embodiment of the present invention, the detected fiducial marker and the template fiducial marker are compared (S610).

In addition, a Jacobian matrix is created on the basis of the difference between the detected fiducial marker and the template fiducial marker (S620).

In addition, the marker pose is optimized so as to minimize the error on the basis of the Jacobian matrix (S630).

In the fine pose refinement step S240 according to the present invention, steps S610 to S630 are repeated up to the appearance image error threshold and as many times as the number of cameras.

In the fine pose refinement step S240 according to the present invention, nonlinear optimization is performed so as to minimize the appearance image error.

The object function E (p) to be minimized can be expressed as follows:

$$E ( p ) = \frac{1}{nm} {\sum }_{i = 0}^{n} {\sum }_{j = 0}^{m} {( I ( {O}_{proj}^{ij} ( p ) ) - T ( {x}^{ij} ) )}^{2} ,$$

where “n” denotes the number of cameras;
“m” denotes the number of detected individual fiducial markers;
“p” denotes a pose vector the marker set (six parameters);
“x^{ij}” denotes an index of the jₜₕ marker out of the detected individual fiducial markers detected from the iₜₕ camera;
“O^{ij}ₚᵣₒⱼ(p)” denotes 2D corner coordinates obtained by reprojecting the 3D corner coordinates of the marker bound with “x” to the pose p;
“I(O^{ij}ₚᵣₒⱼ(p))” denotes a brightness vector of all pixels in a square pixel warped from “O^{ij}ₚᵣₒⱼ(p)”; and
“T(x^{ij})” denotes a brightness vector of all pixels of a template image bound with “x^{ij}”.

According to the present invention, the actual implementation was performed on the basis of the Gauss-Newton algorithm and Backtracking Line Search, the threshold value relating to the amount of the appearance error change was limited to 0.01, and the maximum number of iterations was limited to 10 times.

FIG. 27 shows schematics of the Lucas-Kanade image alignment algorithm.

Referring to FIG. 27, the Lucas-Kanade image alignment is a method of estimating a matrix for batch-transforming the points such that an image formed by specific four points out of a newly captured image becomes most similar to the fiducial image (template) on a nonlinear optimization basis.

The Lucas-Kanade image alignment is performed in the following course:
1) Warp matching target images. Since the fine pose refinement (FPR) step is aimed to precisely track the fiducial marker through matching, the markers recognized from the captured image are warped;
2) Obtain an appearance error image between the warping image and the template image;
3) In order to optimize the captured image, create a first-order derivative image for the X and Y directions of the image;
4) Warp the marker image from the derivative images of each direction, and create the Jacobian matrix for the pose. The original LK image alignment estimates a matrix that transforms 2D points. Therefore, it uses a 4-DOF (degree of freedom) pose as a parameter. However, the FPR uses a 6-DOF pose considering the z-axis as a parameter because it estimates a matrix that transforms 3D points;
5) Multiply the warping images differentiated in each direction by the Jacobian matrix and combine them into a single mage to create the steepest descent image;
6) Multiply the Jacobian matrix obtained in step 4) by the transposed Jacobian matrix to create a pseudo Hessian matrix and obtain the amount of change in the pose;
7) Obtain a change direction of the current value by multiplying the created steepest descent image by the initially obtained error image;
8) Obtain the amount of change in the pose using the calculation result of step 7) and the pseudo-Hessian;
9) Reflect the pose change value obtained in step 8) to the pose; and
10) Repeat steps 2) to 8) until the threshold value or the maximum number of iterations is reached.

Although exemplary embodiments of the present invention have been shown and described, it will be apparent to those having ordinary skill in the art that a number of changes, modifications, or alterations to the invention as described herein may be made, none of which depart from the spirit of the present invention. All such changes, modifications and alterations should therefore be seen as within the scope of the present invention.

What is claimed is:

1. A marker tracking method in a marker tracking system for tracking a marker in augmented reality, the method comprising:
creating marker information necessary for marker pose estimation; and
estimating a marker pose on the basis of the marker information.

2. The marker tracking method according to claim 1, wherein, assuming that an aggregate of one or more individual fiducial markers is called a marker set, the process of creating the marker information includes
creating a fiducial marker by setting the fiducial marker to be used,
creating 3D information of the fiducial marker, and
performing surface calibration for the fiducial marker.

3. The marker tracking method according to claim 2, wherein the process of performing surface calibration for the fiducial marker includes
acquiring a pose of a marker set from each image taken by photographing the marker set at various angles from one or more cameras, and
optimizing the pose of the marker set and poses of individual fiducial markers for each image on the basis of a nonlinear optimization algorithm.

4. The marker tracking method according to claim 1, wherein the process of estimating the marker pose includes
a marker corner information collecting step for collecting information on a corner of the marker,
a marker initial pose estimation step for estimating an initial pose of the marker on the basis of the collected corner information,
a marker coarse pose refinement step for adjusting the initial pose of the marker so as to minimize an error (reprojection error) between 2D corner coordinates of the markers obtained through the marker corner information collecting step and 2D coordinates obtained by reprojecting 3D corner coordinates on the world coordinate system, and
a marker fine pose refinement step for readjusting a detailed pose of the marker on the basis of a dense image alignment technique so as to minimize an appearance difference between the detected fiducial marker image and a template fiducial marker image prepared in advance.

5. The marker tracking method according to claim 4, wherein the dense image alignment technique includes the Lucas-Kanade image alignment.

6. A marker tracking system for tracking a marker in augmented reality, the system comprising:
one or more cameras provided to photograph a marker set as an aggregate of one or more individual fiducial markers; and
a computing device configured to obtain an image captured by the camera, create marker information necessary for estimating a marker pose from the obtained image, and estimate the marker pose on the basis of the marker information.

7. The marker tracking system according to claim 6, wherein the computing device is configured to, in the process of creating the marker information, create a fiducial marker by setting the fiducial marker to be used, create 3D information of the fiducial marker, and perform surface calibration for the fiducial marker.

8. The marker tracking system according to claim 7, wherein the computing device is configured to, in the process of performing surface calibration for the fiducial marker, acquire a pose of the marker set from each image obtained by photographing the marker set at various angles from one or more cameras, and optimize the pose of the marker set and the poses of individual fiducial markers for each image on the basis of a nonlinear optimization algorithm.

9. The marker tracking system according to claim 6, wherein the computing device is configured to, in the process of estimating the marker pose, collect information on corners of the marker, estimate an initial pose of the marker on the basis of the collected corner information, adjust the initial pose of the marker so as to minimize an error (reprojection error) between 2D corner coordinates of the markers obtained through the marker corner information collecting process and 2D coordinates obtained by reprojecting 3D corner coordinates on the world coordinate system, and readjust a detailed pose of the marker on the basis of a dense image alignment technique so as to minimize an appearance difference between the detected fiducial marker image and a template fiducial marker image prepared in advance.

10. The marker tracking system according to claim 9, wherein the dense image alignment technique includes the Lucas-Kanade image alignment technique.
